(19) United States
(12) Patent Application Publication
(10) Pub. No.: US 2014/0350883 A1
(43) Pub. Date: Nov. 27, 2014

(54) Platform for Generating Sensor Data

(71) Applicants: Abraham Carter, Salt Lake City, UT (US); David Scott, North Salt Lake City, UT (US)

(72) Inventors: Abraham Carter, Salt Lake City, UT (US); David Scott, North Salt Lake City, UT (US)

(21) Appl. No.: 14/275,493

(22) Filed: May 12, 2014

Related U.S. Application Data
(60) Provisional application No. 61/822,207, filed on May 10, 2013.

(51) Int. Cl. A61B 5/00; H02J 7/02; G01P 15/00; A61B 5/024; A61B 5/145

(52) U.S. Cl. CPC A61B 5/6802; A61B 5/02444; A61B 5/14542; A61B 5/0015; G01P 15/00; H02J 7/025
USPC 702/141; 702/189

(57) ABSTRACT

A platform is provided for generating, transmitting, and processing sensor data. The platform can provide various components with which a sensor can interface to allow the sensor to provide sensor data using a common mechanism. The platform can include an API which provides a common interface for communicating with a wearable sensor device. The API can allow an application to receive activity recognition and biometric data from one or more sensors in a processed and usable format thereby facilitating the usage of such data. The platform can also comprise a sensor data processor configured to process raw sensor data into a usable format for consumption by other applications, and a sensor data interface for transmitting the raw sensor data to a mobile device for processing by the sensor data processor.

### CROSS-REFERENCE TO RELATED APPLICATIONS

This application claims the benefit of U.S. Provisional Patent Application No. 61/822,207 which was filed on May 10, 2013.

### BACKGROUND

Many devices have been developed for tracking a user's performance during exercise or another activity. For example, GPS watches can track the distance traveled by a user wearing the watch, pedometers can track the number of steps a user takes, and other sensors can be used to measure one or more physiological parameters of the user during an activity. For example, heart rate monitors can detect a user's heart rate, pulse oximeters can detect the saturation of a user's hemoglobin, blood glucose monitors can detect the glucose level in a user's blood, etc.

To track these various parameters, the user is required to wear or carry a specialized device that can both receive raw data from the sensors and process the raw data to generate useful information displayable to the user. For example, many GPS watches are configured to receive raw data from a heart rate monitor (e.g. via Bluetooth) and convert the raw data into an indication of the user's heart rate. Similarly, other devices are configured to receive raw data from a pulse oximeter attached to the user's finger and convert the raw data into an indication of the hemoglobin saturation level in the user's blood.

The requirement that a specialized device be worn or carried can often discourage a user from using such devices. For example, the user may be unable or unwilling to wear a specialized device at all times, and therefore, may be without the device at a time when he desires to measure various physiological parameters. Also, users are often discouraged by the price and complexity of such devices.

### BRIEF SUMMARY

The present invention extends generally to a platform for generating sensor data. By using the platform, many different types and numbers of sensors can be combined into a single system thereby facilitating the tracking of a number of different parameters in a simplified and consistent manner. The platform can provide various components with which a sensor can interface to allow the sensor to provide sensor data using a common mechanism.

In some embodiments, the platform can include an application programming interface (API) which provides a common interface for communicating with a wearable sensor device. The API can allow an application to receive activity recognition and biometric data from one or more sensors in a processed and usable format thereby facilitating the usage of such data.

In some embodiments, the platform can be comprised of a sensor data processor that executes on a mobile device which is configured to process raw sensor data into a usable format for consumption by other applications, and a sensor data interface which can execute on a wearable sensor device for receiving raw sensor data and transmitting the raw sensor data to a mobile device for processing by the sensor data processor.

In some embodiments, the platform can also include a firmware generator the executes on a mobile device which can wirelessly update the firmware on a wearable sensor device to customize the functionality of the device for a particular use or user, and a firmware updater that executes on a wearable sensor device to receive firmware updates and apply the updates to customize the functionality of one or more sensors or other components on the device.

In some embodiments, the platform can also include a Bluetooth low energy (BLE) module for implementing the BLE protocol for communicating sensor data from a wearable sensor device to a mobile device.

In some embodiments, the platform can also include a charging module on a wearable sensor device that enables inductive charging of a battery on a wearable sensor device without interfering with the functionality of any sensors on the wearable sensor device.

This summary is provided to introduce a selection of concepts in a simplified form that are further described below in the Detailed Description. This Summary is not intended to identify key features or essential features of the claimed subject matter.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIGS. 1A and 1B each illustrate various components that can be included in a platform in accordance with one or more embodiments of the invention;

FIG. 2 illustrates an exemplary computer environment 200 in which the platform of the present invention can be used;

FIGS. 3A and 3B represent how different sensors in a wearable sensor device can transmit raw sensor data to mobile device 301 using the common interface provided by sensor data interface;

FIGS. 4A-4C illustrate a process of creating and installing a customized version of firmware on a wearable sensor device based on information obtained about a particular user;

FIGS. 5A-5C illustrate a process of creating and installing a customized version of firmware on a wearable sensor device based on sensor data received from the wearable sensor device;

FIG. 6 illustrates an example configuration of a sensor data processor;

FIGS. 7A and 7B illustrate a simplified example of how matching of raw accelerometer data to sequences of known movement can be performed;

FIG. 8 represents a simplified example of how a user can create a custom entry in a database representing a new or modified movement;

FIG. 9 illustrates a particular example configuration of a portable computing device 101 that can be used to implement the present invention;

FIG. 10 illustrates how a chunk of accelerometer data is converted into a feature set;

FIG. 11 illustrates how a feature set is compared to known feature sets to identify an activity that is being performed; and

FIG. 12 illustrates how multiple feature sets representing the same activity being performed at different speeds can be generated.

### DETAILED DESCRIPTION

The present invention extends generally to a platform for generating sensor data. By using the platform, many different types and numbers of sensors can be combined into a single system thereby facilitating the tracking of a number of different parameters in a simplified and consistent manner. The platform can provide various components with which a sensor can interface to allow the sensor to provide sensor data using a common mechanism.

In some embodiments, the platform can include an application programming interface (API) which provides a common interface for communicating with a wearable sensor device. The API can allow an application to receive activity recognition and biometric data from one or more sensors in a processed and usable format thereby facilitating the usage of such data.

In some embodiments, the platform can be comprised of a sensor data processor that executes on a mobile device which is configured to process raw sensor data into a usable format for consumption by other applications, and a sensor data interface which can execute on a wearable sensor device for receiving raw sensor data and transmitting the raw sensor data to a mobile device for processing by the sensor data processor.

In some embodiments, the platform can also include a firmware generator the executes on a mobile device which can wirelessly update the firmware on a wearable sensor device to customize the functionality of the device for a particular use or user, and a firmware updater that executes on a wearable sensor device to receive firmware updates and apply the updates to customize the functionality of one or more sensors or other components on the device.

In some embodiments, the platform can also include a Bluetooth low energy (BLE) module for implementing the BLE protocol for communicating sensor data from a wearable sensor device to a mobile device.

In some embodiments, the platform can also include a charging module on a wearable sensor device that enables inductive charging of a battery on a wearable sensor device without interfering with the functionality of any sensors on the wearable sensor device.

### Example Computer Architecture

Embodiments of the present invention may comprise or utilize special purpose or general-purpose computers including computer hardware, such as, for example, one or more processors and system memory, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system.

Computer-readable media is categorized into two disjoint categories: computer storage media and transmission media. Computer storage media (devices) include RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other similarly storage medium which can be used to store desired program code means in the form of computer-executable instructions or data structures and which can be accessed by a general purpose or special purpose computer. Transmission media include signals and carrier waves.

Computer-executable instructions comprise, for example, instructions and data which, when executed by a processor, cause a general purpose computer, special purpose computer, or special purpose processing device to perform a certain function or group of functions. The computer executable instructions may be, for example, binaries, intermediate format instructions such as assembly language or P-Code, or even source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like.

The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices. An example of a distributed system environment is a cloud of networked servers or server resources. Accordingly, the present invention can be hosted in a cloud environment.

### Platform for Generating Sensor Data

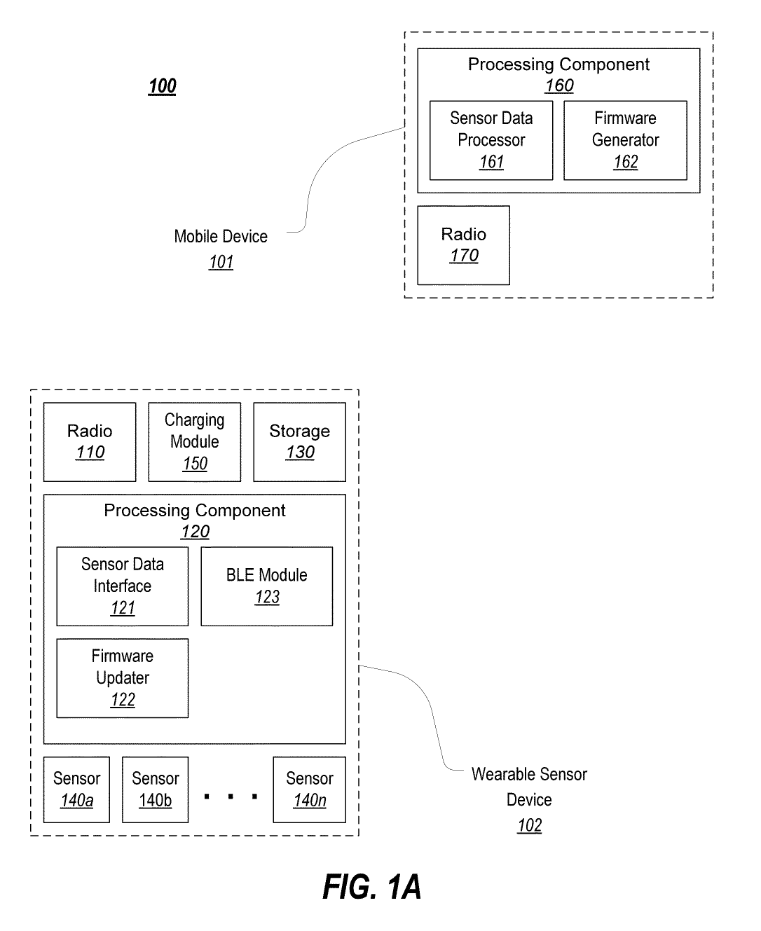

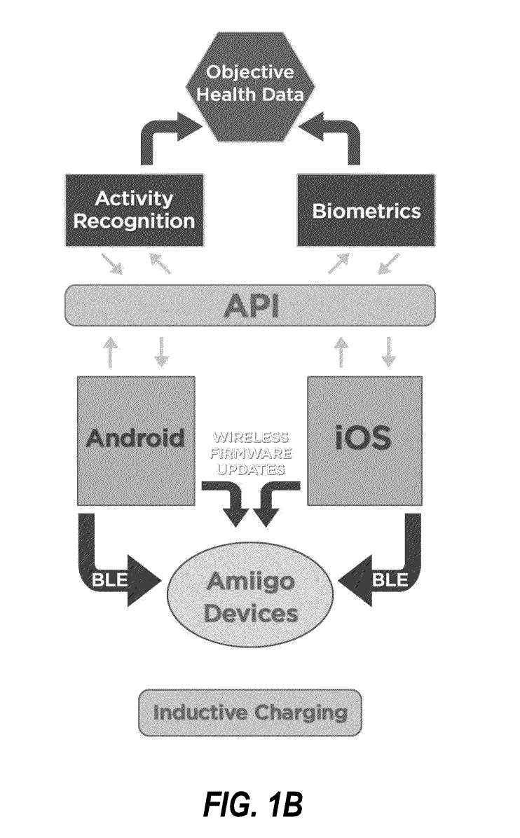

FIG. 1 illustrates an example of various components or functionality that can be included in a platform 100 for generating sensor data in accordance with one or more embodiments of the invention. FIG. 1 illustrates a mobile device 201 and a wearable sensor device 102. Wearable sensor device 102 can include one or more sensors (e.g. sensors 140a-140n) for generating sensor data that is transmitted by wearable sensor device 102 to mobile device 201 for processing.

Platform 100 can include various components that can be utilized on a wearable sensor device to facilitate the generation and processing of sensor data. For example, wearable sensor device 102 can include a sensor data interface 121 which sensors 140a-140n use to provide sensor data for transmission to mobile device 201. Sensor data interface 121 can provide a common interface for receiving sensor data from a number of different types of sensors. This common interface facilitates the use of different types of sensors within a wearable sensor device because the common interface abstracts, from the sensor, how sensor data is transmitted to mobile device 201.

Platform 100 can also include a Bluetooth low energy (BLE) module 123 for execution on wearable sensor device 102. BLE module 123 can implement the BLE protocol on wearable sensor device 102 when transmitting sensor and other data to mobile device 201. In this way, the battery life of wearable sensor device 102 can be extended.

Platform 100 can also include a firmware updater 122 which can be used to update the firmware of any of sensors 140a-140n. Platform 100 can also include a firmware generator 162 which executes on mobile device 201 to generate customized firmware updates for sensors 140a-140n. The customized firmware updates can customize the functionality of a particular sensor based on how wearable sensor device 102 is or will be used, or based on one or more characteristics of a user of wearable sensor device 102.

Platform 100 can also include a charging module 150 for controlling the charging of a battery on wearable sensor device 102. Charging module 150 can comprise hardware and/or software for facilitating the charging of the battery. In some embodiments, charging module 150 can comprise components for allowing wearable sensor device 102 to be charged inductively. For example, the components can include an induction coil. Charging module 150 can comprise the particular layout of the components that allows for inductive charging while still allowing one or more other sensors to be incorporated into wearable sensor device 102.

For example, some sensors may require the use of an LED and light sensor that must be positioned near the exterior surface of wearable sensor device 102. For example, when a pulse oximeter is included in wearable sensor device 102, charging module 150 can be configured with an induction coil that is positioned near an exterior surface of wearable sensor device 102 (to allow for efficient induction charging) while still allowing the LED and light sensor of the pulse oximeter to be positioned near an exterior surface.

Platform 100 can also include a sensor data processor 161 that executes on mobile device 201 to process raw sensor data received from wearable sensor device 102 into usable sensor data. Because sensor data interface 121 allows virtually any type of sensor to transmit raw data to mobile device 201, sensor data processor 161 can be configured to receive the raw data and transform it into a usable format that can be consumed by other applications. In this way, an application can receive usable sensor data from virtually any type of sensor without needing to know any sensor specific data format or protocol. In other words, sensor data processor 161 can be configured to transform raw sensor data received from many different types of sensors and in many different formats into a common format easily understood by other applications. This allows many different applications to easily integrate into platform 100 to receive and utilize sensor data from virtually any type of sensor.

FIG. 1B provides a simplified diagram of how the platform of the present invention can facilitate the creation of objective health data using a mobile device (e.g. an Android phone or iPhone). The depicted API can represent the components of the platform that reside on mobile device 101 (which may be an Android, iPhone, or other type of mobile phone or mobile device). This API allows simplified integration with wearable sensor device (represented as Amiigo Devices in FIG. 1B) to receive activity recognition and biometric data about a user.

For example, a third party provider can build an application that employs the API to receive the activity recognition and biometric data generated from virtually any type of sensor incorporated into a wearable sensor device. The API allows this data to be represented in a usable format that abstracts the sensor specific raw data from the applications. As such, the activity recognition and biometric data generated by the API can serve as a common language representing objective health data that may be consumed by a variety of applications of different types (e.g. health monitoring applications, activity performance applications, insurance-based applications, etc.).

As also shown, the platform can include the necessary components on the mobile device and the wearable sensor device for enabling the use of BLE for transmitting data between the devices. Many current implementations of the BLE protocol are difficult to interface with. The platform can provide a simplified implementation (or SDK) for the BLE protocol that is accessible via the API which facilitates an applications use of the BLE protocol for communicating with a wearable sensor device.

Further, as illustrated in FIG. 1B, the platform can include the necessary components for causing wireless firmware upgrades to be made. The platform can also be configured to report the occurrence of such updates via the API to an application to allow the application to better customize initial versions of firmware that may be provided on a mobile device or a wireless sensor device.

Finally, as illustrated in FIG. 1B, the platform can include the necessary components for allowing the wearable sensor device to be charged using inductive charging. These components, as well as the other components that reside on the wearable sensor device, can facilitate the creation of third party wearable sensor devices. For example, a third party provider could more easily build a wearable sensor device that includes various sensors by utilizing the communication components, charging components and update components provided by the platform. The platform would therefore reduce development time and complexity and provide a common platform to enhance the interchangeability of sensor devices.

### Facilitating Transmission of Raw Sensor Data and Generation of Usable Data Using the Platform's Sensor Data Interface and Sensor Data Processor

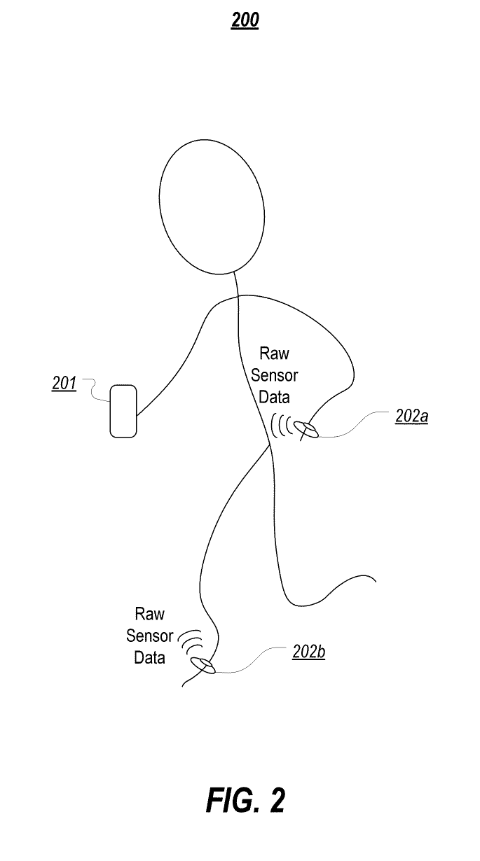

FIG. 2 illustrates an exemplary computer environment 200 in which the platform of the present invention can be used. Computer environment 200 includes a mobile device 201 and wearable sensor devices 202a, 202b that are worn by a user during a workout or other activity. Mobile device 201 can contain the same or similar components of the platform as shown in mobile device 101. In particular, mobile device 201 can include a sensor data processor 161 and a firmware generator 162. In a typical implementation, mobile device 201 can be a user's smart phone or other device capable of running a mobile application (e.g. an MP3 player or tablet).

Wearable sensor devices 202a, 202b can be configured with the same or similar components of the platform as shown in wearable sensor device 102. For example, each of wearable sensor devices 202a, 202b can include one or more different types of sensors for detecting various parameters. In a particular embodiment, the sensors can include an accelerometer, a blood glucose sensor, a pulse oximeter, a skin temperature sensor, or a blood pressure sensor among others. Each wearable sensor device 202a, 202b can include a sensor data interface 121 for transmitting raw sensor data received from the one or more sensors of the wearable sensor device to mobile device 201. Similarly, each wearable sensor device 202a, 202b may also include one or more of a charging module 150, a BLE module 123, or a firmware updater 122. Specifically, not all wearable sensor devices need contain each component of the platform (e.g. if it is not desired that a wearable sensor device provide all the functionality provided by the platform).

An accelerometer in a wearable sensor device can be used to detect specific movements of the user's body parts on which the wearable sensor device is worn. For example, in the particular embodiment shown in FIG. 2, wearable sensor device 202a is worn around the user's wrist (e.g. as a bracelet) and includes an accelerometer for determining the specific motion the user's arm makes during a workout. In such embodiments, wearable sensor device 202a can also include one or more sensors for detecting one or more of the user's physiological parameters during the workout.

Similarly, wearable sensor device 202b, as shown in FIG. 2, can be worn on or around the user's foot or ankle and provide accelerometer data representing the specific motion of the user's leg during the workout. Wearable sensor device 202b can also contain one or more sensors for detecting various physiological parameters.

In each wearable sensor device, sensor data interface 121 can receive raw sensor data from each of the sensors (e.g. an accelerometer and a blood glucose sensor) in the wearable sensor device, and transmit the raw sensor data to mobile device 201 for processing by the sensor data processor 161. In this way, sensor data interface 121 can provide a common interface for virtually any type of sensor thereby facilitating the inclusion of the different types of sensors within a single wearable sensor device.

Although FIG. 2 depicts two wearable sensor devices 202a, 202b being worn around the wrist and on the foot respectively, the present invention is not limited to any specific number of sensors or any particular placement of the sensors on the user's body. For example, one or more wearable sensor devices can be worn on the elbow, hip, knee, head, etc.

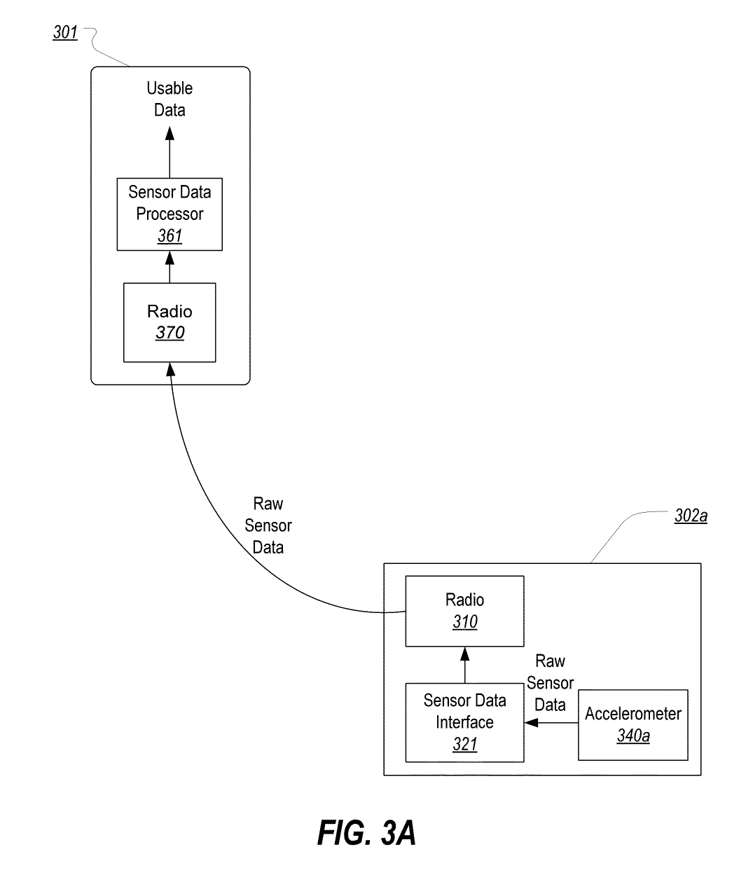

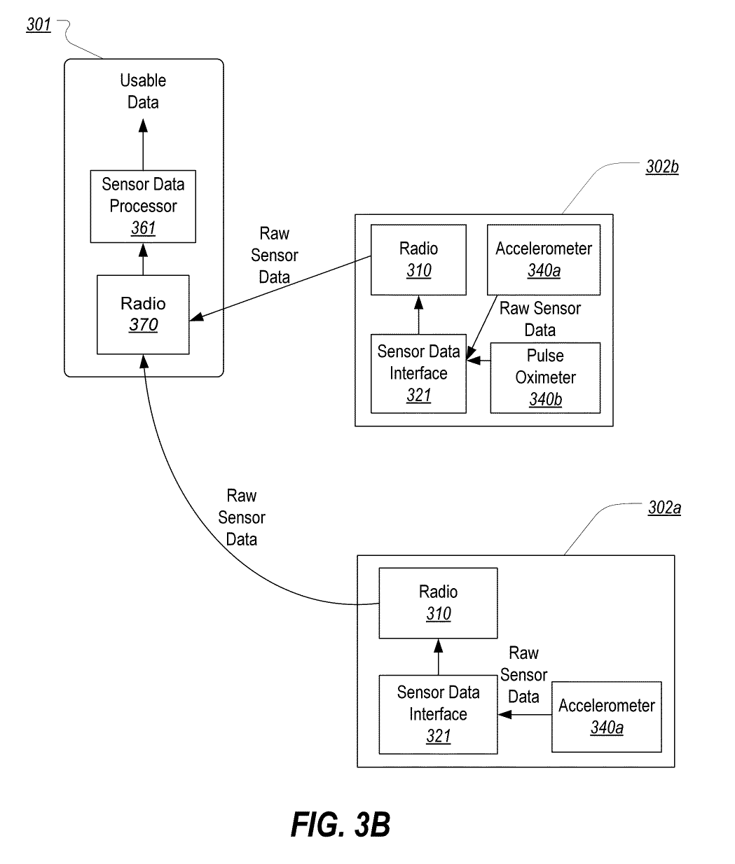

FIGS. 3A and 3B represent how different sensors in a wearable sensor device can transmit raw sensor data to mobile device 301 using the common interface provided by sensor data interface 321. As shown in FIG. 3A, sensor 302a includes an accelerometer 340a. Raw sensor data (i.e. raw accelerometer data) output by accelerometer 340a is received at sensor data interface 321 which performs the necessary functionality to allow the raw sensor data to be transmitted to mobile device 301 via radio 310. Also, in some embodiments where the wearable sensor device also includes a BLE module 123, sensor data interface 321 can employ the functionality of BLE module 123 to cause the transmission of the raw sensor data to be carried out using the BLE protocol.

FIG. 3B is similar to FIG. 3A but also shows that wearable sensor device 302b includes an accelerometer 340a and a pulse oximeter 340b which both generate raw sensor data that is provided to sensor data interface 321 for transmission to mobile device 301 as described above. In this manner, sensor data interface 321 provides a common interface to different types of sensors that abstracts from the sensors the functionality required for transmitting the raw sensor data to mobile device 301. This abstraction facilitates the inclusion of different types of sensors within a single wearable sensor device. Accordingly, by using the platform of the present invention, a diverse number and configurations of wearable sensor devices can be more easily designed.

In some embodiments, the functionality provided by sensor data interface can include determining how frequently raw sensor data should be transmitted. This can be done to conserve battery power. For example, sensor data interface 321 can identify when raw sensor data should be transmitted immediately to mobile device 301 (e.g. when real-time data is desired, when it is determined that mobile device 301 is within range, etc.), or when raw sensor data should be stored for later transmission (e.g. when the raw sensor data is not changing significantly between sensor readings, when it is desired to conserve battery power (which determination may be made in conjunction with BLE module 123 in some cases), etc.). This type of functionality can be incorporated into the platform (and more specifically, into sensor data interface 123) to allow sensors to be built on top of the platform without requiring the sensors to implement or even understand how the functionality is provided.

One benefit of providing the platform for use by wearable sensor devices is that third party vendors can easily create custom wearable sensor devices. For example, a third party vendor desiring to provide a wearable sensor device that includes an accelerometer, a pulse oximeter, and a heart rate monitor can incorporate the components of the platform on the wearable sensor device so that each of the sensors only needs to provide raw sensor data to the sensor data interface. As can be seen, the third party can simply configure each of the sensors to provide raw sensor data to the sensor data interface while allowing the platform to handle the necessary functionality for transmitting the raw sensor data to a mobile device in an appropriate and efficient manner.

Similarly, sensor data processor 361 on mobile device 301 can facilitate the design of applications or other modules that consume sensor data. Sensor data processor 361 can be configured to understand raw sensor data received from many different types of sensors and to process the different types of raw sensor data into a format that an application or other module can easily consume. In some embodiments, sensor data processor 361 can process and format raw sensor data into one or more common formats. In this manner, an application can consume sensor data by simply understanding the common format or formats without needing to understand the particular format or structure of the raw sensor data produced by the sensor. As such, the platform can facilitate the design of many different applications that can consume sensor data for a variety of purposes such as for displaying the sensor data to a user, generating metrics of a user's or group of user's performance, creating a database of generated sensor data for data mining, etc.

In some embodiments, the processing performed by sensor data processor 361 (or alternatively, sensor data interface 321) can include correlating raw sensor data from multiple sensors. For example, when a user is wearing more than one wearable sensor device, or when a wearable sensor device includes more than one sensor, it may be desirable to correlate the raw sensor data produced by each sensor.

In a particular example, a user may desire to know what his pulse oximeter readings are at particular times during performance of an activity. To facilitate this type of correlation, sensor data processor 361 (or alternatively, sensor data interface), can correlate timestamps associated with the raw sensor data generated by each sensor to ensure that raw sensor data generated at the same time is compared. This processing can be performed by sensor data processor 361, by sensor data interface 321, or by both components. For example, sensor data interface 321 may associate timestamps with the individual raw sensor data and sensor data processor 361 may correlate the timestamps.

Further, sensor data processor 361 can provide functionality for identifying particular patterns, identifiers, or other markers within raw sensor data. The usable data generated by sensor data processor 361 can therefore include indications of the identified patterns, identifiers, or markers. In one example, the identification of patterns can include identifying patterns in raw accelerometer data representing specific motions performed by a user. Sensor data processor 361 can then include an indication of the specific motion performed in the usable data. This process of detecting specific motions from raw accelerometer data is further described below in the section titled “Processing Raw Accelerometer Data To Identify Particular Movements.”

Similarly, sensor data processor 361 can identify patterns or identifiers in sensor data representing physiological readings. For example, sensor data processor 361 can identify a plateau in a user's blood oxygen level and generate an indication that the plateau represents the user's VO₂ₘₐₓ. Further, sensor data processor 361 can correlate this plateau with raw sensor data to provide more information such as indicating the rate at which a motion is being performed when the plateau is reached based on correlated accelerometer data, indicating a heart rate when the plateau is reached based on correlated heart rate data, indicating a blood glucose level when the plateau is reached based on correlated blood glucose data, etc.

By identifying such patterns in raw sensor data and generating usable data representing the occurrence of such patterns, sensor data processor 361 facilitates the consumption of sensor data. In this specific example, an application desiring the monitor a user's VO₂ₘₐₓ can be designed much more easily because the application can consume the usable data that identifies that occurrence of the VO₂ₘₐₓ along with any other correlated patterns or identifiers in other sensor data rather than having to be configured to process raw sensor data to identify such occurrences within the application itself.

### Customizing Firmware of a Sensor for a Particular Use by Using the Platform's Firmware Generator and Firmware Updater

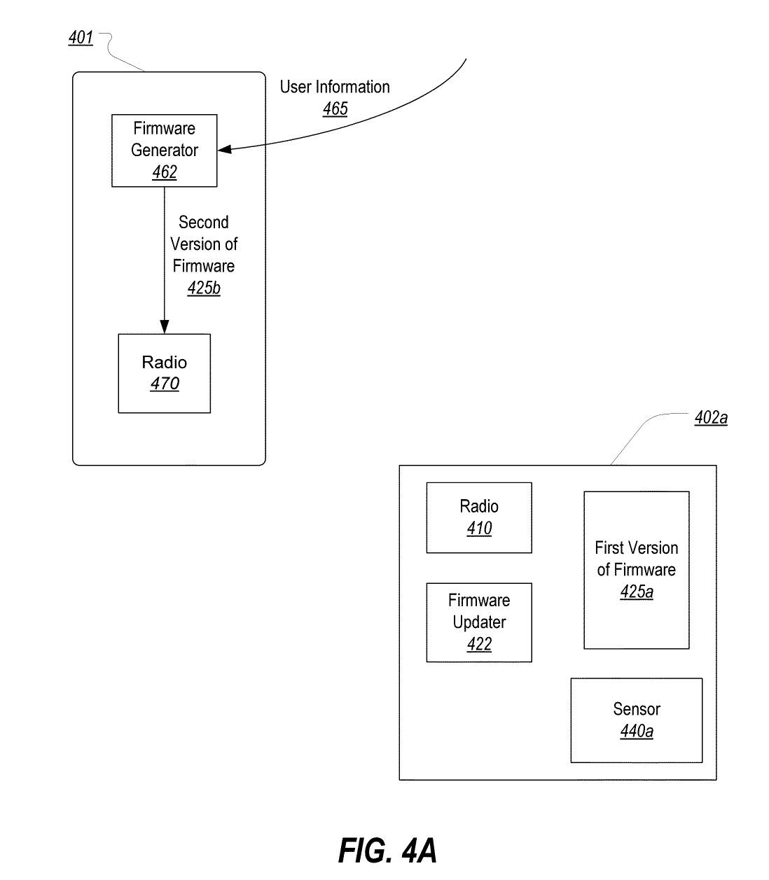

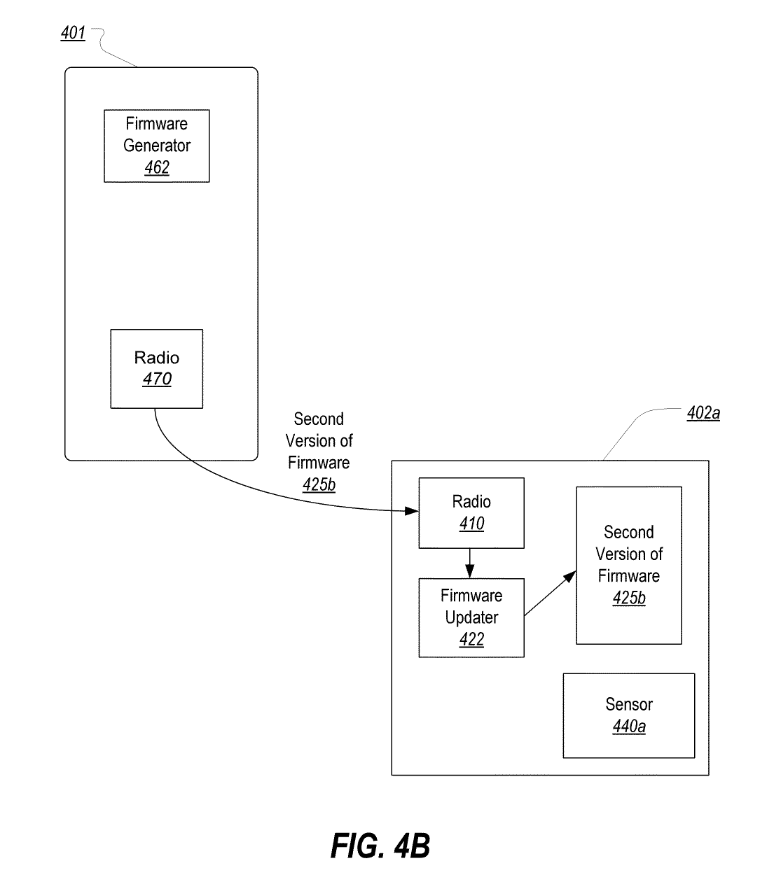

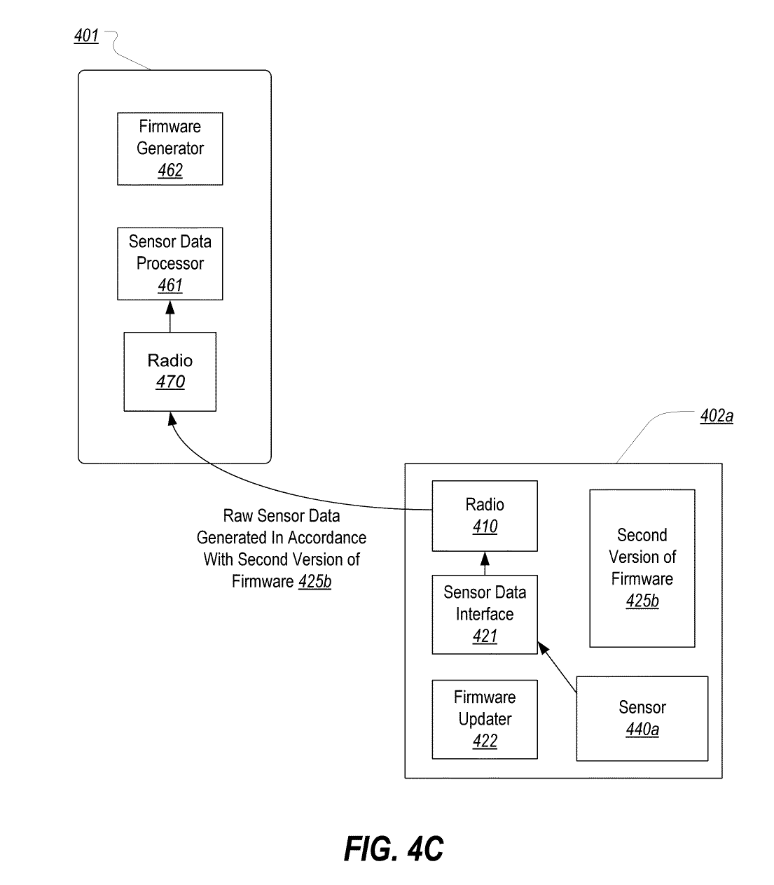

In some embodiments, the platform of the present invention can allow a mobile device to customize firmware on a wearable sensor device to cause the wearable sensor device (or more particularly, the sensor or sensors on the device) to function in a manner that is customized for the particular way in which the wearable sensor device is used or intended to be used. For example, as shown in FIG. 4A, mobile device 401 can be configured to communicate wirelessly with wearable sensor device 402a (e.g. via Bluetooth) to update firmware of sensor 440a. Updating the firmware in this manner enables the sensor 440a to be customized for a particular user or a particular use of wearable sensor device 402a.

The firmware generator of the platform can be configured to identify that the current version of the firmware on a wearable sensor device is not optimal for how the wearable sensor device is being used, is intended to be used, or for the particular user that is using the wearable sensor device. For example, FIG. 4A shows that firmware generator 462 receives user information 465. User information 465 can comprise many different types of information such as characteristics of the user that is using wearable sensor device 402a, preferences of the user, indications of how wearable sensor device 402a will be used or is being used, etc. Firmware generator 462 can also know what version of the firmware is installed on wearable sensor device 402a (e.g. by wearable sensor device 402a communicating such information to mobile device 401).

Using user information 465, firmware generator 462 can determine whether the first version of firmware 425a that is currently installed on wearable sensor device 402a is optimal. When firmware generator 462 determines that customizations can be made to the first version of firmware 425a, it can generate a second version of firmware 425b that includes the customizations to optimize the performance of wearable sensor device 402a.

As shown in FIG. 4B, the second version of firmware 425b has been transmitted by mobile device 401 to wearable sensor device 402a. Firmware updater 422 on wearable sensor device 402a receives the second version of firmware 425b and installs it on wearable sensor device 402a. In this way, the second version of firmware 425b causes the functionality of sensor 440a to be customized based on user information 465 received by firmware generator 462. For example, as shown in FIG. 4C, with the second version of firmware 425b installed on wearable sensor device 402a, sensor 440a generates raw sensor data in accordance with the second version of firmware 425b which is provided to sensor data interface 421 for transmission to sensor data processor 461.

Although this example depicts the functionality of sensor 440a being customized, the same process can be used to update firmware for customizing the functionality of another sensor or a component of the platform. Various examples of the types of customizations that can be made to firmware on a wearable sensor device are provided below.

In some embodiments, a sensor (or a component of the platform) may initially contain firmware (e.g. when sold) that causes the sensor to perform in a manner that would be most effective for an average person. However, because each individual user may use the sensor differently, the initial firmware may not provide the most optimized functionality for a particular user or use.

As an example, a wearable sensor device that comprises an accelerometer may be sold with firmware that optimizes the functionality of the accelerometer when used for running. These optimizations, however, may cause the accelerometer to be less optimized when used for yoga. Because of this, a user that intends to use or has used the wearable sensor device to track yoga movements may not receive optimized movement tracking from the accelerometer. To address these types of scenarios, the present invention can identify how a particular user intends to use or has used a wearable sensor device, create a firmware update that is customized for the particular user's use of the wearable sensor device, and wirelessly transmit the customized firmware update to the wearable sensor device. In this way, each individual wearable sensor device can be customized for the particular user that is using or will use the wearable sensor device.

As another example, a wearable sensor device that comprises a pulse oximeter may be sold with firmware that optimizes the functionality of the pulse oximeter for a user having average skin density. However, if the user's skin has an increased density (and therefore requires a stronger intensity of light for the sensor to adequately work), the firmware can be adjusted so that a stronger light is emitted. Such determinations can be made during or after use of the pulse oximeter (e.g. by identifying that data received from the pulse oximeter is deficient), or prior to use (e.g. by receiving user input that indicates that a stronger light intensity may be desired).

Customizations to the firmware of a wearable sensor device can be based on many different factors (i.e. user information 465 can represent many different types of information). For example, prior to using a wearable sensor device, a user may complete a registration process during which the user provides information specifying the user's characteristics (e.g. height, weight, age, resting heart rate, etc.) and the intended uses of the wearable sensor device. Based on such information, a customized update to the firmware of the wearable sensor device can be created which tailors the functionality of the wearable sensor device to provide a more optimal experience for the user when wearing the wearable sensor device.

Similarly, after the user has begun using the wearable sensor device, he may provide additional information or modified information that can be used to identify customizations that can be made to the firmware. For example, if a user initially specified that he intended to use the wearable sensor device during running, but later decided to use the wearable sensor device during swimming, the user can provide such input to allow a firmware update to be created that is customized for the user's use of the wearable sensor device while swimming.

In addition to creating firmware updates in response to user input, a customized firmware update can also be created automatically by detecting that sensor data received from the wearable sensor device indicates that the wearable sensor device is not functioning in an optimal manner. For example, it can be determined that a particular sensor is taking readings too frequently or not frequently enough based on the particular way in which it is being used. In such cases, the firmware can be updated to decrease the sample rate (e.g. to conserve battery power), or to increase the sample rate (e.g. to more accurately identify a particular movement the user is performing).

In some embodiments, a firmware update can be made to customize how a wearable sensor device uses memory (e.g. storage 130) or a radio (e.g. radio 110). For example, based on how the wearable sensor device is used or is intended to be used, it could be determined that the wearable sensor device will always be within range of the mobile device to which the wearable sensor device transmits sensor data.

For example, a user may specify that he will always carry his mobile phone while wearing a wearable sensor device, or the mobile phone may determine that it is always in proximity of the wearable sensor device during use of the wearable sensor device. In such cases, there may never be a need for the wearable sensor device to store sensor date prior to transmitting the sensor data to the mobile device. Therefore, a customized firmware update can be generated and transmitted to the wearable sensor device that causes the wearable sensor device to continuously transmit sensor data to the mobile device without first storing the sensor data in memory.

Examples where customizing the firmware so that sensor data is not stored in memory include when the user carries his phone while performing the exercise (e.g. while jogging, biking, hiking, golfing, etc.), when the user wears the wearable sensor device while exercising on a stationary device (e.g. a treadmill) with the mobile device nearby, when the user wears the wearable sensor device to monitor a movement or other parameter that is performed in a single location with the mobile device nearbly (e.g. while sleeping, while lifting weights, while doing yoga, etc.).

Similarly, if the mobile device is never or rarely within range of the wearable sensor device during use (meaning that the sensor data must be stored until the mobile device is within range), a customized firmware update can be generated that causes the radio of the wearable sensor device to be turned off during use of the wearable sensor device. In this way, the wearable sensor device will not needlessly attempt to transmit data when the mobile device is not within range. In some cases, the radio can be customized to be turned off throughout the use of the wearable sensor device, or may be customized to make periodic scans to determine when the mobile device may be in range. Examples where customizing the firmware so that the radio is turned off during use include when the wearable sensor device is used during swimming (because the user probably will not carry his phone while swimming), or when the user does not desire to carry the mobile device while performing an exercise that requires traveling outside the range of the radio.

Similar customizations can be made to BLE module 123. For example, BLE module 123 can be configured to implement the BLE protocol or the standard Bluetooth protocol. Customizations can be made to BLE module 123 to cause the most optimal protocol to be used, or to cause a particular protocol to be used at certain times based on certain factors.

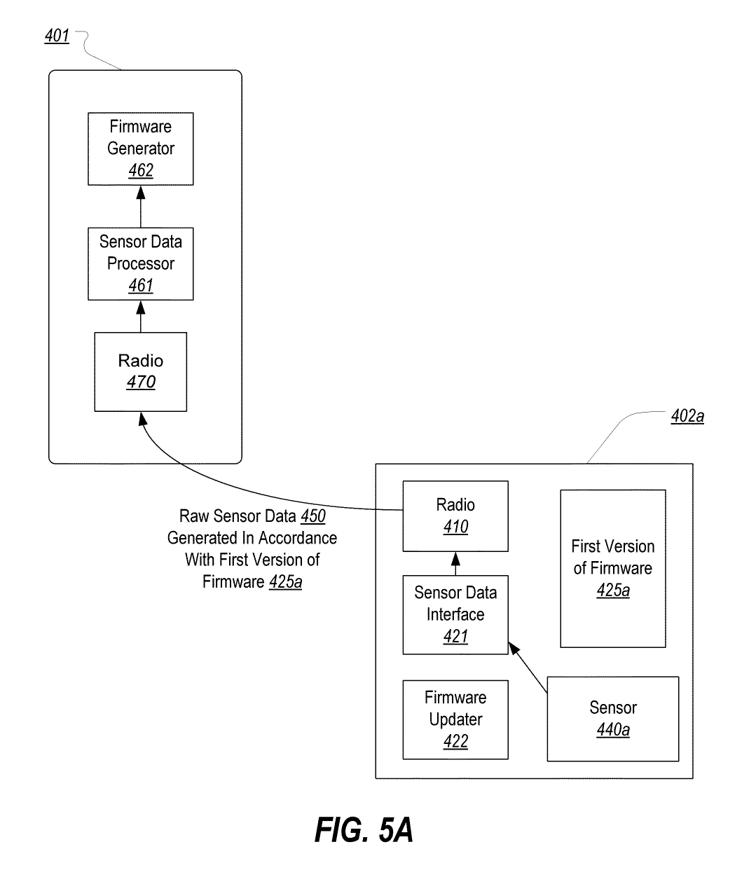

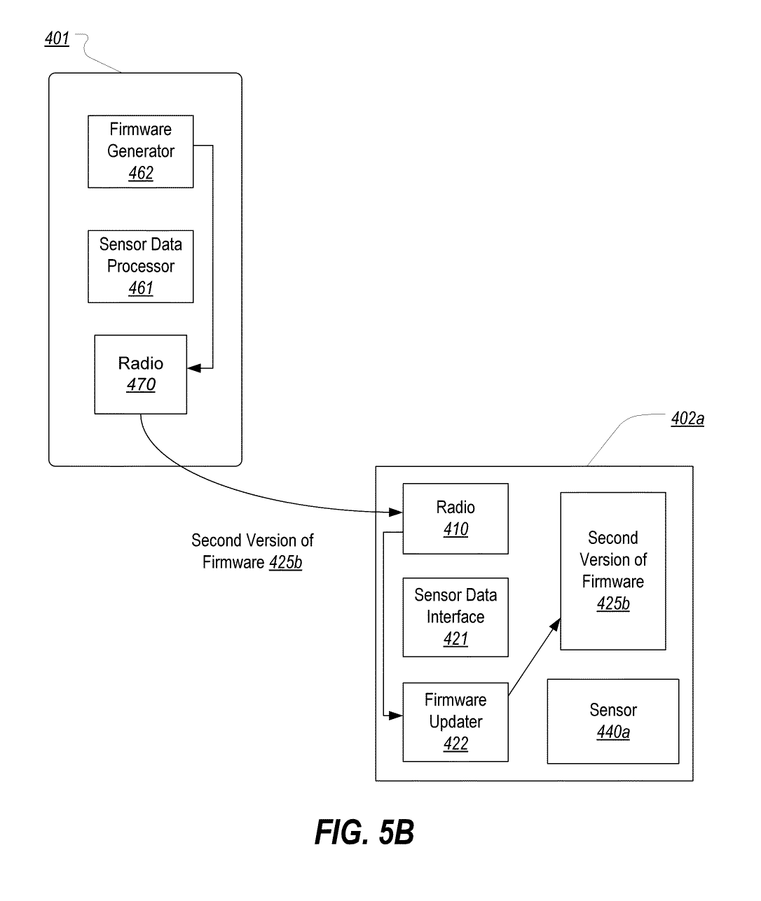

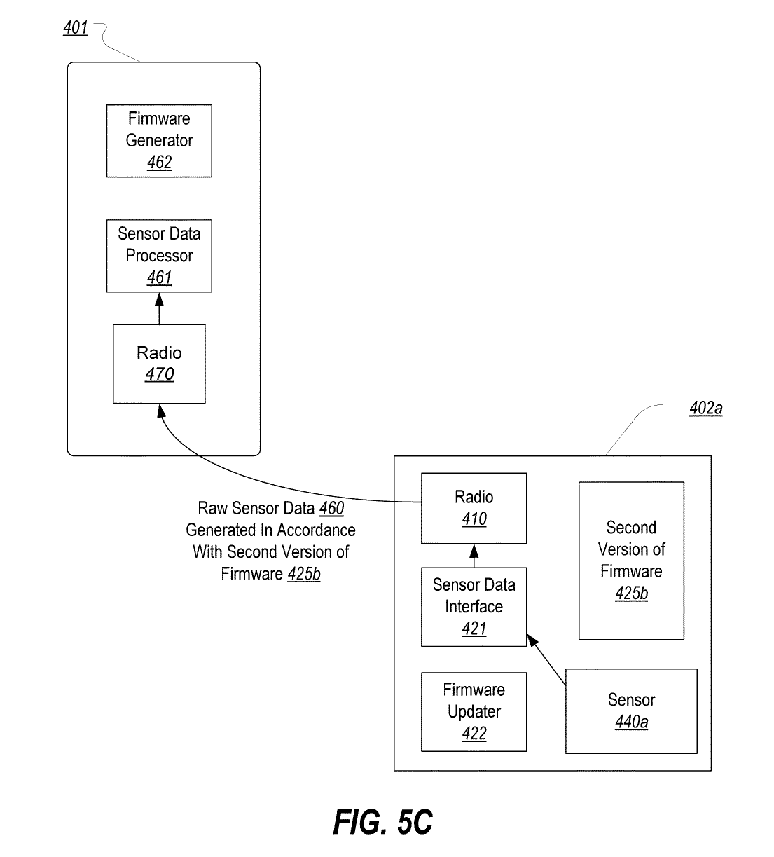

FIGS. 5A-5C illustrate an example where the customization to the firmware is based on a determination that the raw sensor data generated while a first version of firmware 425a is installed on wearable sensor device 402a indicates that sensor 440a is not performing optimally for the particular way in which wearable sensor device 402a is being used. Although wearable sensor device 402a is shown as having a single sensor 440a, the process depicted in FIGS. 5A-5C can be used when a wearable sensor device includes more than one sensor. In other words, a customized firmware update can modify the functionality of one or more sensors in a wearable sensor device.

In FIG. 5A, wearable sensor device 402a is shown as having a first version of firmware 425a for controlling sensor 440a. The first version 425a can be an initial version supplied with wearable sensor device 402a or an already updated version transmitted to wearable sensor device 402a. FIG. 5A also shows that mobile device 401 receives data 450 generated by sensor 440a in accordance with the first version of firmware 425a. For example, data 450 can comprise accelerometer data (when sensor 440a is an accelerometer), hemoglobin saturation data (when sensor 440a is a pulse oximeter), blood glucose levels (when sensor 440a is a blood glucose monitor), or any other type of data generated by a sensor that can be incorporated into wearable sensor device 402a to provide meaningful data. It is also noted that data 450 can comprise a combination of data from multiple sensors. In other words, if wearable sensor device 402a includes multiple sensors, data 450 can include sensor data generated by any number of the multiple sensors.

Firmware generator 462 can determine from data 450 that the first version of firmware 425a which is currently installed on wearable sensor device 402a for controlling sensor 440a is not optimal. In response, as shown in FIG. 5B, firmware generator 462 can generate a second version of firmware 425b which is customized for the particular way in which the user is using wearable sensor device 402a (as indicated by data 450). These customizations can include modifying a sampling rate of sensor 440a or modifying another controllable parameter of sensor 440a (which will be different depending on the specific type of sensor).

In FIG. 5B, mobile device 401 is shown as wirelessly transmitting the second version of firmware 425b to wearable sensor device 402a which replaces, updates, or otherwise modifies first version 425a. Then, as shown in FIG. 5C, with the second version of firmware 425b installed on wearable sensor device 402a, sensor 440a generates data 460 in accordance with the second version of firmware 425b (e.g. using a customized sampling rate, a customized power level, a customized data set, etc.). Accordingly, data 460 generated after the second version of firmware 425b is installed can be optimized for the particular way in which the user is using wearable sensor device 402a.

In some embodiments, in addition to customizing the firmware on the wearable sensor device, firmware generator 462 (or another separate component of the platform) can customize sensor data processor 461. For example, because a wearable sensor device can include sensors that may provide a substantial amount of data related to a large number of exercises or movements, sensor data processor 461 needs the capability to process the large amount of sensor data it may receive. However, in most cases, a particular user will only use a wearable sensor device to monitor a subset of movements or parameters that sensor data processor 461 is capable of identifying/tracking.

In such cases, sensor data processor 461 can be customized based on how the user is using or intends to use the wearable sensor device. For example, if the user intends only to use the wearable sensor device to track the performance of pushups and the hemoglobin saturation level during the pushups, sensor data processor 461 can be customized to process such data more efficiently. In this example, sensor data processor 461 may be customized by deactivating the other movements that sensor data processor 461 is configured to identify (e.g. running movements, biking movements, swimming movements, etc.) which may result in sensor data processor 461 running more efficiently leading to quicker response times and reduced battery consumption.

Deactivating a movement can refer to causing sensor data processor 461 to not compare sensor data of an unknown movement to the known data representing a particular activity. For example, if a database of known movements stores data representing the sensor data generated when each of one hundred different movements is performed, sensor data processor 461 can be configured to only compare unknown sensor data generated by the wearable sensor device to the stored data representing the active movements. In this way, when the user performs a movement that is active, sensor data processor 461 can potentially identify the movement using many fewer comparisons than when all movements are active because comparisons will not be made to the stored data representing deactivated movements.

Similarly, in some cases, the user may intend to use the wearable sensor device to identify a custom movement. In such cases, sensor data processor 461 can be customized by creating and activating the custom movement while deactivating some or all of the other movements for which sensor data processor 461 stores data. Accordingly, the user experience can be optimized by both customizing the firmware on the wearable sensor device as well as customizing sensor data processor 461 on the user's mobile device.

As stated above, one type of customization that can be made to the firmware is configuring the sampling rate of one or more sensors to provide optimized data given a particular activity the user will perform. For example, a preinstalled version of the firmware can cause an accelerometer to generate accelerometer readings at a first sampling rate. This first sampling rate may be optimal for many common uses of the wearable sensor device.

However, a particular user may desire to use the wearable sensor device to monitor a movement for which a different sampling rate would be more optimal. As an example, the preinstalled version of the firmware may be optimized for exercises such as jogging, biking, or swimming where the speed of movement of the user's arm or leg is relatively slow. In contrast, the particular user may desire to use the wearable sensor device to monitor a golf swing or to track sprinting where the user's arm or leg is likely moving at a much higher speed.

In such cases, either by receiving user input identifying the intended use of the wearable sensor device or by detecting the actual use of the wearable sensor device while performing the desired movement, firmware generator 462 can determine that a higher sampling rate would produce better accelerometer data for monitoring the golf swing or the sprinting motion. Accordingly, a customized update to the firmware can be provided to optimize the performance of the accelerometer during the golf swing or sprinting motion.

Also, to emphasize how the firmware can be customized for a particular user (and not just for a particular use), it is noted that one user's golf swing may require a different sampling rate for optimal performance than another user's golf swing. In such cases, an initial customized version of the firmware may be provided to each user's wearable device that is customized to monitor a golf swing. Then, after detecting the accelerometer data generated during the user's golf swing (or in response to user input), firmware generator 462 may further customize the firmware for the particular user's golf swing.

Similarly, if one user intends to use a wearable device only for a first activity while another user intends to use a wearable device for a first and a second activity, different customized versions can be generated for each device. For example, in the first case, the sampling rate can be customized to be optimal for the first activity. In contrast, in the second case, the sampling rate can be customized to provide the best possible performance over the first and second activities (e.g. when each activity has a different optimal sampling rate).

In some embodiments, the particular version of firmware that is provided to a wearable sensor device can be generated by selecting from among a number of customizations available. For example, depending on the type of sensor being controlled, there may be various settings for controlling the sensor with various options that can be set for each setting. Depending on the information about the particular user, one or more settings for a sensor can be configured with the appropriate option to provide customized firmware that is most optimal for the particular user.

Referring to the sampling rate example above, there may be a number of different values to which a sensor's sampling rate can be set. The appropriate sampling rate for a particular user can be selected and specified in the customized version of the firmware for that particular user. Similarly, any other settings for the sensor or another sensor can be selected from among the various available options and specified in the customized version of the firmware. In this way, logic can be used to select the appropriate values for each configurable setting of a sensor based on the information known about a particular user.

In some embodiments, firmware updater 422 on a wearable sensor device can be configured to automatically detect that its firmware is not optimal for the manner in which a particular user is using the wearable sensor device. In such cases, firmware updater 422 can automatically update its firmware to provide more optimal performance. For example, a wearable sensor device can initially be configured to use a sampling rate of 100 Hz. During use, firmware updater 422 can determine that a sampling rate of 50 Hz is sufficient for the types of activities the particular user performs while wearing the wearable sensor device. In response, firmware updater 422 can automatically update its firmware so that a sampling rate of 50 Hz is used.

Similarly, in some embodiments where firmware updater 422 is capable of automatically detecting that firmware is not optimal, firmware updater 422 can be configured to cause firmware updates on other wearable sensor devices. For example, when firmware updater 422 on a bracelet sensor device updates firmware to use a sampling rate of 50 Hz, it may also cause another wearable sensor device (e.g. a shoe clip sensor device) to be updated with customized firmware for sampling at the 50 Hz rate. In this way, the firmware updater 422 on the bracelet sensor device can automatically customize the firmware on the bracelet as well on the other wearable sensor device based on observations of how a particular user is using the devices.

In some embodiments, firmware updater 422 on one wearable sensor device can be used to update the firmware of another wearable sensor device with an update received from a mobile device. For example, firmware updater 422 on a bracelet sensor device can be configured to receive firmware updates from a mobile device (e.g. a mobile phone). The firmware updates can pertain to the bracelet sensor device or to another sensor device such as a shoe clip sensor device. In such cases, firmware updater 422 on the bracelet sensor device can be configured to route the firmware update to the shoe clip sensor device so that the firmware on the shoe clip sensor device can be customized for a particular user.

An example of where firmware updater 422 on one wearable sensor device can be used to update the firmware of another wearable sensor device is when one device is more powerful or has more capabilities than another device. Referring to the example above, a bracelet sensor device may be configured with the ability to wirelessly receive firmware updates from a mobile device while the shoe clip sensor device may not. In such cases, the bracelet sensor device and shoe clip sensor device can be provided with physical contact points which allow a direct transfer of firmware updates from the bracelet sensor device to the shoe clip sensor device. In this way, many wearable sensor devices can be updated with customized firmware without requiring each device to have the circuitry to wirelessly receive such updates from the mobile device. This can result in wearable sensor devices that are smaller and require less battery power.

To summarize, one benefit of the firmware generator and the firmware updater of the platform is that they can facilitate the customization of a wearable sensor device automatically. A third party provider can build a wearable sensor device on the platform knowing that the performance of the wearable sensor device can be automatically customized and optimized for a particular use or user without needing to provide functionality to create or install such updates. For example, if a third party provider includes a custom sensor in a wearable sensor device, the third party provider may need only inform the firmware generator and/or the firmware updater of the customizable parameters of the custom sensor. Then, the firmware generator and firmware updater can automatically detect customizations that should be made and install such customizations. In this way, the third party provider's device can provide an enhanced and customized user experience without burdening the third party provider to provide such enhancements or customizations on a per device basis.

### Processing Raw Accelerometer Data to Identify Particular Movements

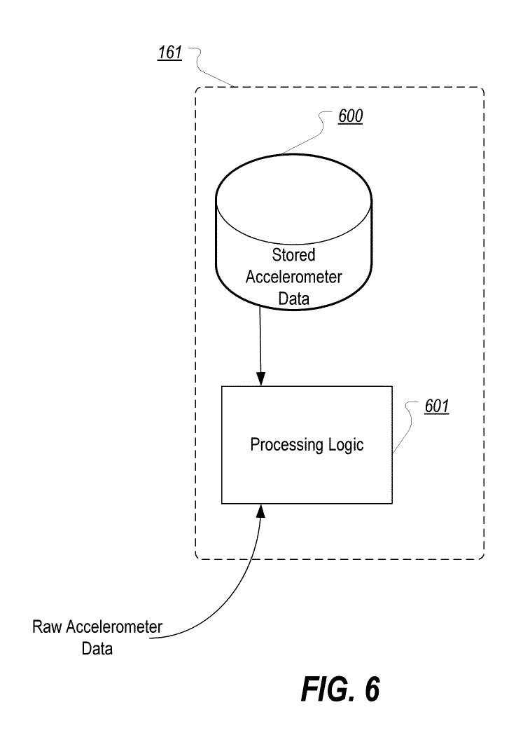

FIG. 6 illustrates an example configuration of sensor data processor 161. As shown, sensor data processor 161 contains processing logic 601 and a database 600. Processing logic 601 is configured to receive accelerometer data from one or more accelerometers (e.g. accelerometers 740a, 740b). Database 600 contains stored accelerometer data representing particular movements. When processing logic 601 receives raw accelerometer data from one or more accelerometers, processing logic 601 can access the stored accelerometer data in database 600 to attempt to match the received accelerometer data to the stored accelerometer data representing a particular movement. If a match is found, processing logic 601 can store an indication that the user has performed the particular movement.

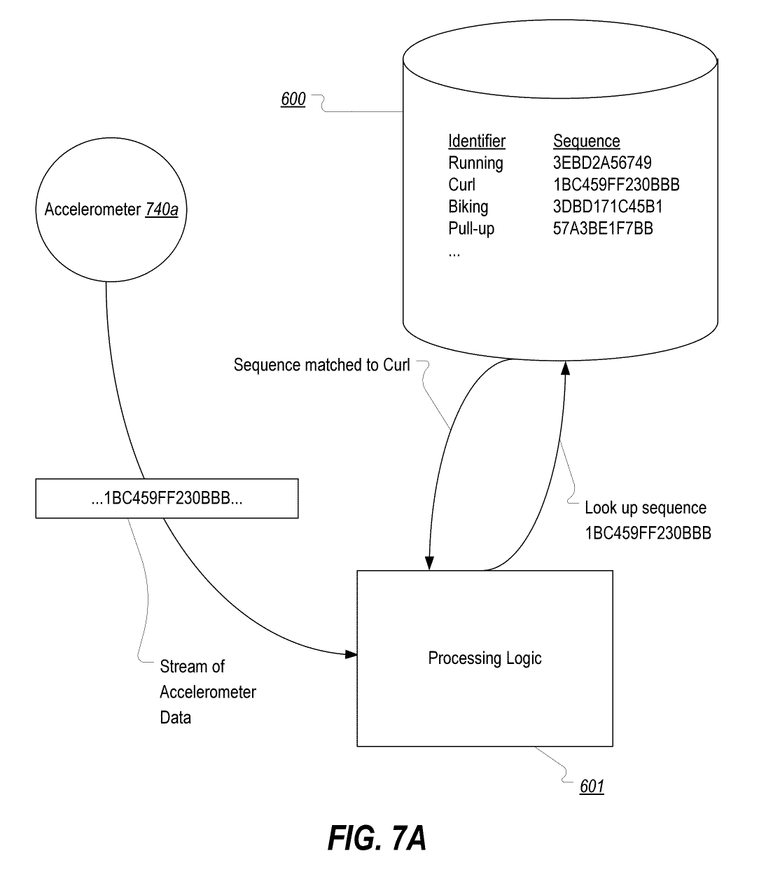

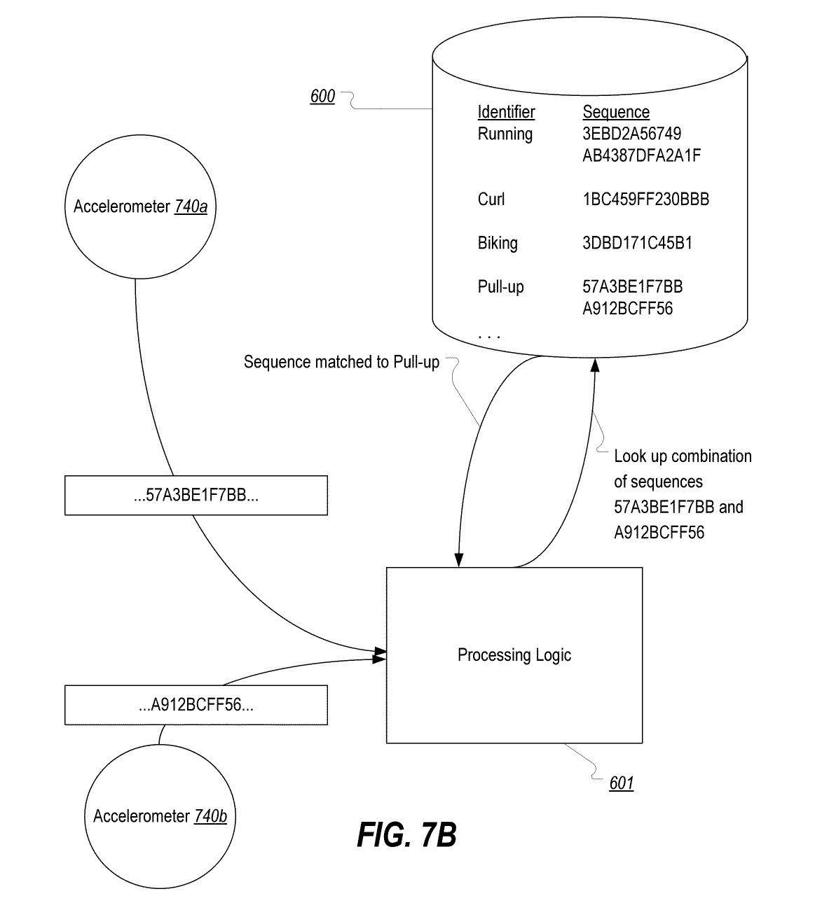

FIGS. 7A and 7B illustrate a simplified example of how this matching can be performed. This simplified example illustrates the direct matching of accelerometer data to known sequences of accelerometer data. However, as further described below, this matching can also be performed by first processing the raw accelerometer data into a more useful format for comparison.

FIG. 7A shows a single accelerometer transmitting a stream of accelerometer data which is received by processing logic 601. Database 600 is shown as storing various sequences with an associated identifier. Database 600 can be built using known sequences that identify a particular movement.

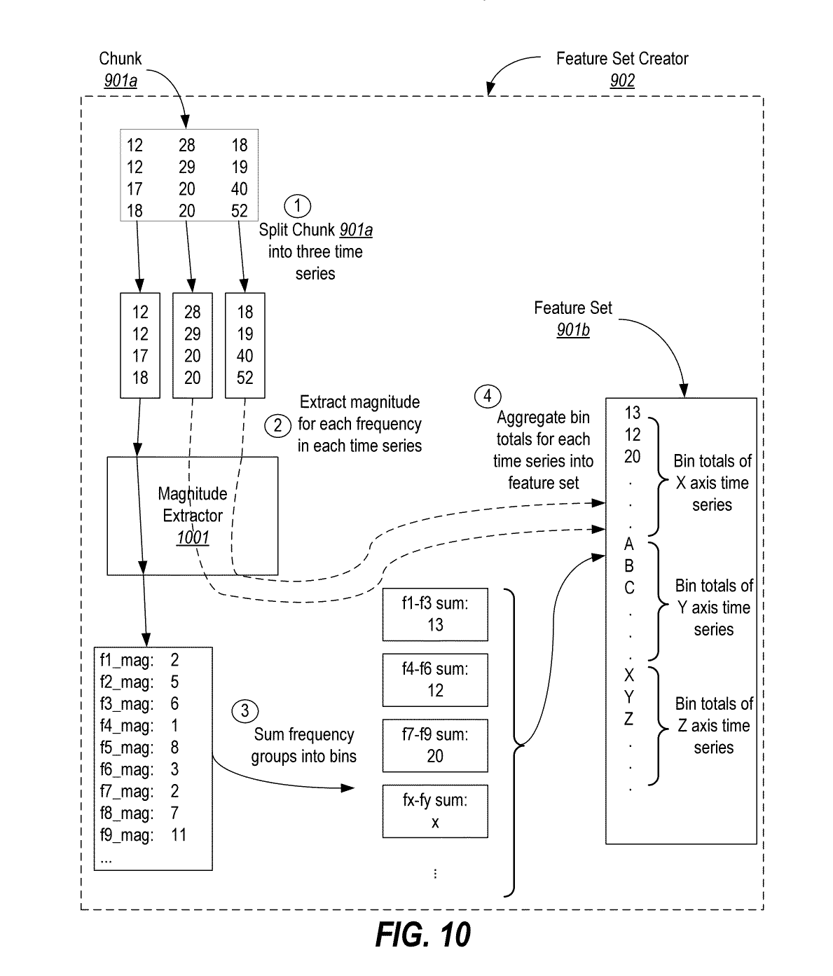

As mentioned, the depicted sequences in database 600 are simplified to better illustrate the process by which the matching occurs. However, in many cases, rather than a single sequence, a range of sequences or even logic that identifies whether a received sequence matches a particular movement can be used. One specific example of how the data can be stored in database 600 is provided below with respect to FIGS. 10-12.

When processing logic 601 receives or identifies a sequence in the accelerometer data from accelerometer 740a (which in this example is 1BC459FF230BBB), processing logic 601 can perform a look-up to identify whether the received sequence matches any stored sequence in database 600. Because the received sequence matches the stored sequence corresponding to Curl, processing logic 601 can know that the user has performed a curl. Once it is determined that the user has performed a curl, processing logic 601 can perform various actions such as updating a count of the number of curls performed, updating a display, generating a notification, etc.

FIG. 7B is similar to FIG. 7A but represents in simplified form how a particular movement can be determined using accelerometer data from multiple accelerometers. As shown, both accelerometers 740a and 740b are transmitting accelerometer data representing the movement of the user's body on which each accelerometer is placed.

As in FIG. 7A, processing logic 601 receives the accelerometer data and performs a look-up. In contrast to FIG. 7A, database 600 is shown as storing two sequences for some movements. For example, the entry for Running includes two sequences which represent the typical motion of an accelerometer attached to the user's shoe and of an accelerometer attached to the user's wrist. The entry for Pull-up likewise includes two sequences.

It is noted that, although only two sequences are shown, it is possible to use more than two sequences for some movements (e.g. when an exercise involves distinct movement of more than two body parts). Also, for some exercises, accelerometer data from only one accelerometer may be required to identify the exercise even if the user is wearing multiple accelerometers. For example, because the user's foot generally remains stationary during a curl (and because it may not be necessary or desirable to identify leg movement during a curl), only one sequence may be stored that represents accelerometer data from an accelerometer on the user's wrist, hand, or arm.

Whether processing logic 601 receives data from one, two, or more accelerometers, processing logic 601 can perform a look-up using any or all of the received data. In FIG. 7B, the look-up is performed using sequences 57A3BE1F7BB and A912BCFF56 which match the stored sequences for a pull-up. Accordingly, processing logic 601 knows that the user has performed a pull-up and can respond accordingly. In another example, if the received accelerometer data had included sequences of 3DBD171C45B1 and 1276BBB34, the look-up could have matched only one of the sequences (the first sequence) which would have identified that the user is pedaling a bike.

In some embodiments, the particular movements identified in database 600 can be highly granular. For example, database 600 can include an entry identifying Running and an entry identifying Running While Pushing A Jogging Stroller. In this example, the entries may contain the same or similar sequence representing the motion of the user's leg while running (e.g. identifying a step), and a sequence representing the motion of the user's arm in each case (e.g. swinging in the case of Running, and stationary in the case of Running While Pushing A Jogging Stroller). Similar distinctions can be made with other types of exercises (e.g. a standard pull-up versus a cross-fit type swinging pull-up).

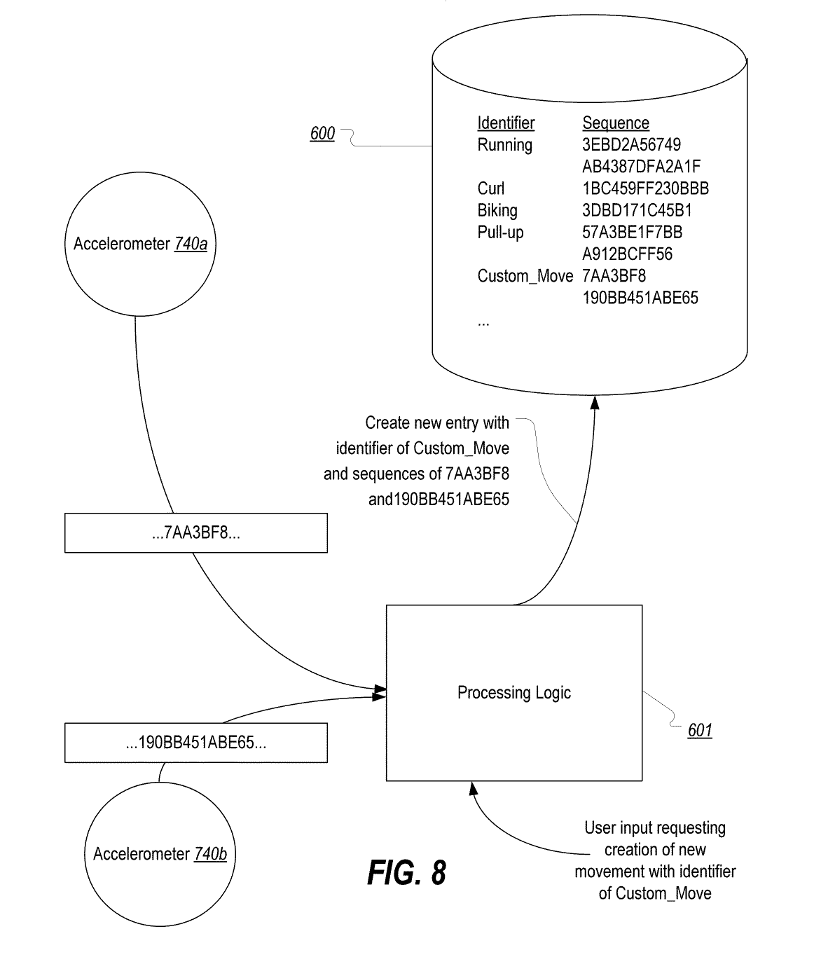

FIG. 8 represents a simplified example of how a user can create a custom entry in database 600 representing a new or modified movement. For example, if database 600 does not include an entry for a particular yoga movement, the user can provide input to processing logic 601 requesting that a custom entry be created. This can be done by identifying the accelerometer data received from one or more accelerometers while the custom movement is being performed, identifying a sequence within the accelerometer data, and storing the sequence in database 600 with an associated identifier (which may be provided by the user).

FIG. 8 shows that sequences received from both accelerometers 740a and 740b (7AA3BF8 and 190BB451ABE65) are stored in database 600 with an identifier of Custom_Move. Sensor data processor 161 can provide a common interface that allows another application or the user to provide input requesting the creation of a custom movement. Accordingly, an application can be built on the platform of the present invention to provide the ability to create custom movements. The application need only understand how to request the creation of a custom movement but need not understand how the custom movement is actually created.

In some embodiments, once a user has created a custom entry in database 600, the custom entry can be shared with other users. For example, if a user has performed a custom cross-fit movement and desires to challenge his friends to perform the same movement, a custom entry created for the movement can be sent to the friends' portable computing devices (either directly or via a central server). Sensor data processor 161 (or another component of the platform) can provide functionality for sharing custom movements.

In this manner, database 600 can store entries for virtually any movement. For example, database 600 can initially be supplied with a number of common movements and can later be updated either by receiving user created custom entries or by receiving new entries received from a central server or other users' devices.

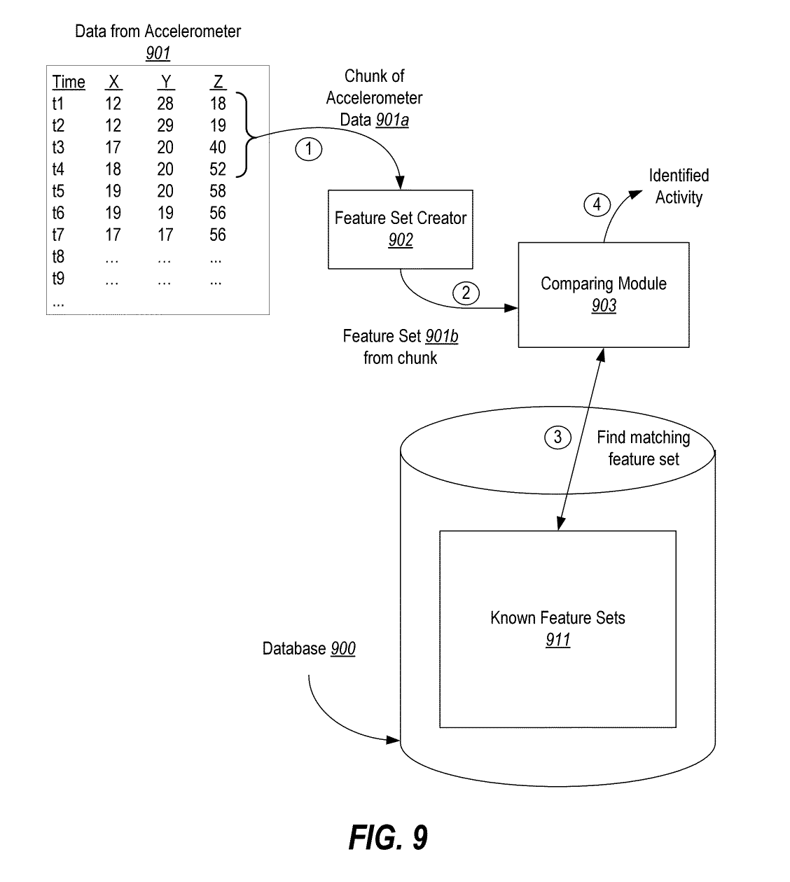

FIG. 9 illustrates a particular example configuration of sensor data processor 161 (e.g. example components of processing logic 601) that can be used in one or more embodiments of the platform. The numbers used to describe accelerometer data in FIGS. 9-12 are exemplary and have been chosen to simplify the description of the invention. However, the specific format used in the described processes can vary as desired.

FIG. 9 shows an example data stream 901 that is received from an accelerometer. In this example, it is assumed that a single accelerometer is being used. However, similar processing can be performed on the data received from one or more other accelerometers.

FIG. 9 represents a general description of the process of converting the data 901 into a feature set that is compared to known feature sets to identify an activity being performed. Data 901 is shown as comprising three axes (x, y, z) of accelerometer readings over a number of time periods (t1-t9).

The first step of the depicted process is to divide data 901 into various chunks. The division of data 901 into chunks can be based on many factors. In the example shown in FIG. 9, a chunk 901a is shown as comprising the accelerometer data received during the time interval t1-t4 (e.g. 4 seconds if a time period is 1 second, 2 seconds if a time period is 0.5 seconds, etc.). The second step comprises feature set creator 902 generating a feature set 901b from chunk 901a. The third step comprises comparing module 903 comparing feature set 901b to the known features sets 911 in database 900 to determine the known feature set that most closely matches feature set 901b. This closest matching feature set represents the activity being performed by the user. Finally, the fourth step comprises outputting the name of this identified activity.

FIG. 10 illustrates a more detailed example of how a chunk is converted into a feature set. As shown, chunk 901a is first split into three time series: one for the x axis data, one for the y axis data, and one for the z axis data in chunk 901a. In some embodiments, each time series is then further processed independently of the other time series in the chunk. FIG. 10 shows how steps 2 and 3 are applied to the time series for the x axis data while the processing for the y and z axis data is shown simply with dashed lines for clarity. However, in other embodiments, the processing performed on each time series can be interdependent on other times series. Interdependent processing can facilitate consistent scaling of the data to nondimensional speed.

Step 2 involves extracting the magnitude of various frequencies in the data of each time series. In some embodiments, this can be accomplished by applying a Fourier transform to the data, although other techniques could also be used. In FIG. 10, the extracted magnitudes for 9 frequencies (f1_mag-f9_mag) are shown as an example, although other numbers of frequencies could equally be used.

Once the magnitudes of the various frequencies are determined, step 3 involves summing groups of the magnitudes into bins. In FIG. 10, these bins are shown as the sum of the magnitudes of the first through third frequencies, the sum of the fourth through sixth frequencies, the sum of the seventh through ninth, frequencies, etc.

Bins covering different combinations of frequencies can also be used. Also, in some embodiments, no bins (or a bin containing the magnitude of a single frequency) can be used. One benefit of using bins is that the larger the number of frequencies whose magnitudes are summed into a bin, the simpler the processing. However, simplifying the processing by using larger bins reduces the accuracy of the process. Therefore, depending on a particular embodiment, the bin size can be selected to maximize processing efficiency without unreasonably affecting accuracy.

Finally, at step 4, the sums from the bins are aggregated to form feature set 901b. The feature set includes the sums from the bins generated for each time series (i.e. for the y and z time series as well as the x time series) as indicated by the dashed lines from the y and z time series. Accordingly, after this process, feature set 901b is in a format that enables the comparison of accelerometer data to the known feature sets stored in the database.

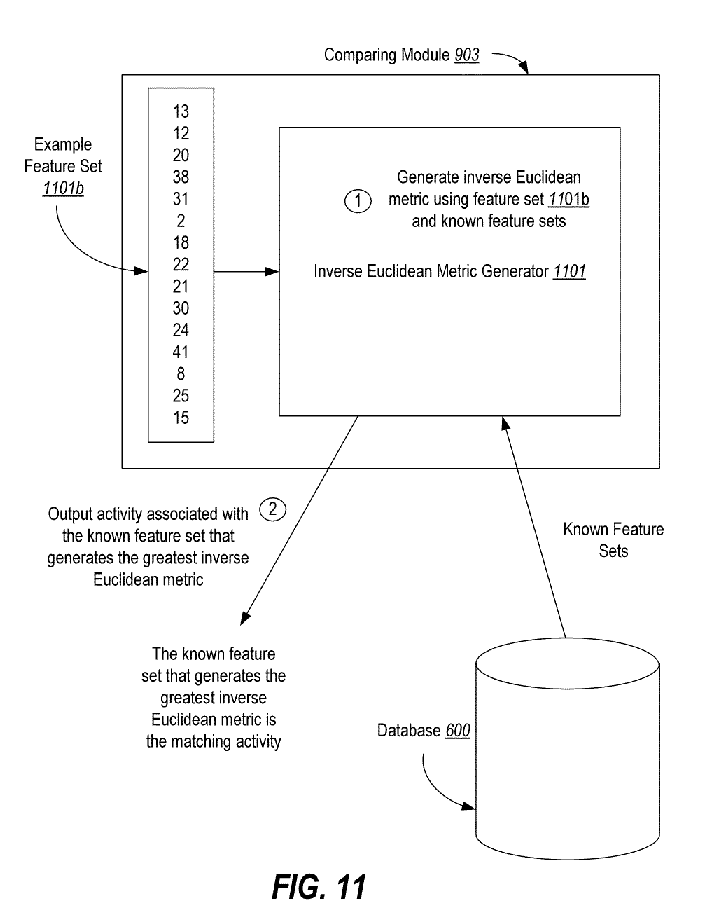

FIG. 11 represents an example of how comparing module 903 can compare feature set to the known feature sets to identify an activity being performed. As shown, this comparison can be made using the inverse Euclidean metric of the feature set and each known feature set. The inverse Euclidean metric can be generated from two feature sets using the following function:

$$\frac{1}{\sqrt{{\sum }_{1}^{i} {( ( featureSet [ i ] ) - ( referenceSet [ i ] ) )}^{2}}}$$

where i is an index to the bin total, featureSet is the feature set generated from the current received accelerometer data, and referenceSet is the feature set of a known activity. In some embodiments, this function can include a frequency-specific weighting factor such as:

$$\frac{1}{\sqrt{{\sum }_{1}^{i} ( ( weight [ i ] ) * {( ( featureSet [ i ] ) - ( referenceSet [ i ] ) )}^{2} )}}$$

Once the inverse Euclidean metric has been generated for each combination of the feature set and the known feature sets, the inverse Euclidean metric having the greatest value is selected, and the activity associated with the known feature set used to generate the greatest value is output as the matched activity.

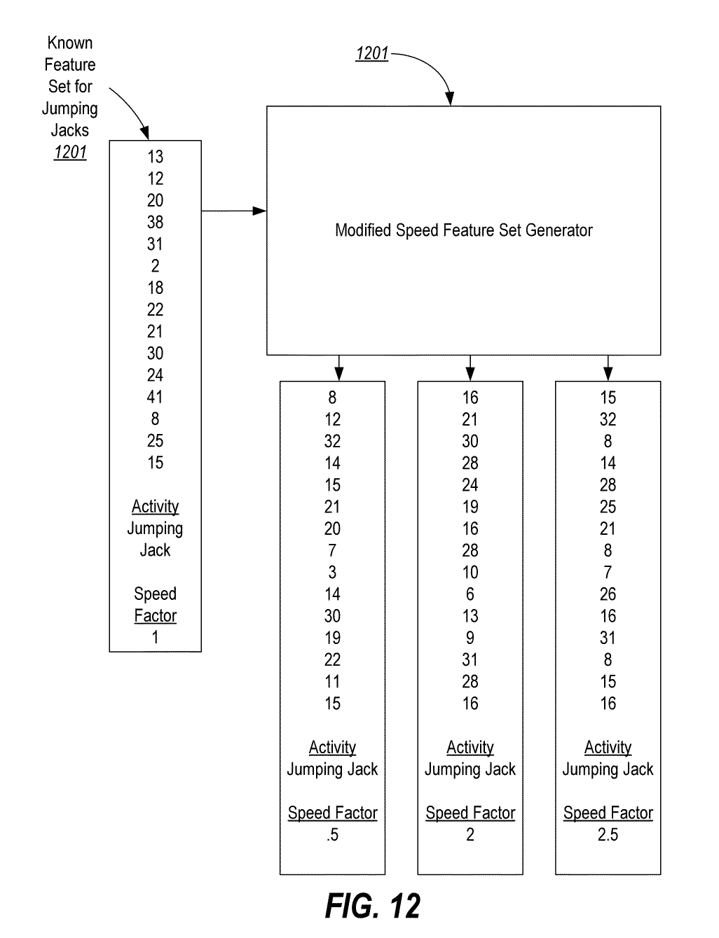

In some embodiments, to ensure that a particular activity can be matched independent of the speed at which the user is performing the particular activity, multiple feature sets can be generated for the particular activity. FIG. 12 illustrates how this can be done. Known feature set 1201 represents the feature set generated when jumping jacks are performed at a standard speed. Known feature set includes an indication of the type of activity with which it is associated (i.e. Jumping Jack), and a speed factor which in this case is 1. In other words, the numbers listed in feature set 1201 represent the accelerometer data that would be generated when a user is performing jumping jacks at a standard speed.

FIG. 12 includes a modified speed feature set generator 1201 which can generate other feature sets from feature set 1201. Modified speed feature set generator 1201 can generate new feature sets from an existing feature set by modifying the time basis of the input accelerometer data (e.g. by a factor ranging from less than one to greater than one). As shown, three new feature sets have been generated that each represent the jumping jack activity, but correspond to the performance of jumping jacks at a different speed. Specifically, the new feature sets correspond to jumping jacks being performed at half speed, double speed, and two-and-half speed with respect to the reference speed 1 of feature set 1101.

In this example, database 600 can store all four feature sets thereby allowing sensor data processor 161 to identify not only the specific activity being performed, but the speed at which the activity is being performed. Specifically, because the numbers (i.e. bin totals) in each feature set corresponding to a particular activity will vary (as shown in FIG. 12), the feature set that yields the highest inverse Euclidean metric will be selected as the matching activity. The speed factor (as well as the activity) listed in the matching feature set can be accessed to determine how fast the user is performing the particular activity. In some embodiments, a feature set can also include information regarding the degree of cyclic motion among the frequencies it contains.

Although not shown, when more than one accelerometer is being used, more than one feature set can be used in the comparison step. Using the jumping jack example, accelerometer data from an accelerometer on the user's wrist and from an accelerometer on the user's foot may be required to appropriate detect that a jumping jack (or a specific type of jumping jack) is being performed. In such cases, the process depicted in FIG. 10 can be performed independently on both sets of accelerometer data thereby generating two feature sets. These two features sets can be input to the comparing module 903 which can compare both feature sets to known feature sets.

For example, a known feature set for jumping jacks may actually contain two feature sets (one for the wrist data and one for the foot data). Comparing module 903 can determine whether the input feature sets match both feature sets in the known feature set.

Alternatively, this process of matching multiple feature sets can be performed independently. For example, comparing module 903 can determine which known feature set matches the feature set representing the wrist data, and independently determine which known feature set matches the feature set representing the foot data. Once two matched feature sets have been found, a lookup can be performed to determine whether a known activity exists that includes both identified known feature sets (e.g. a known activity for jumping jacks may contain both identified known feature sets). In other words, in this scenario, the movement of the arm is identified separately from the movement of the foot, and then the two identified movements are used to determine whether a known activity exists that includes both movements.

Correlating Sensor Data Obtained from a Wearable Sensor Device with Sensor Data Obtained from Sensors of a Mobile Device

In some embodiments of the invention, the platform can enable a mobile device to correlate sensor data received from a wearable sensor device with sensor data received from one or more sensors of the mobile device. For example, sensor data processor 161 can receive raw sensor data from one or more sensors on a wearable sensor device (e.g. via sensor data interface 121) as well as from one or more sensors on the mobile device on which sensor data processor 161 is implemented.

For example, a mobile device can include a microphone for detecting audible sounds that may occur while the user is sleeping. Similarly, a mobile device can include a light sensor (e.g. the light sensor used to control the screen brightness of a smart phone) for detecting the presence of light while the user is sleeping. Also, a mobile device can include a camera for capturing an image or series of images of the user while the user is sleeping. In such cases, sensor data processor 161 (or another component that is in communication with sensor data processor 161) can correlate the two types of sensor data to provide an indication of why a user performs some action during sleep.

For example, when a user moves his arm while sleeping, an accelerometer within a wearable sensor device attached to the user's arm can generate sensor data representing the movement of the user's arm. This sensor data can be transmitted by the wearable sensor device to the mobile device. Additionally, a microphone within the mobile device can detect a sound and generate sensor data representing the occurrence of the sound.

Sensor data processor 161 (or another component in communication with sensor data processor 161) can process the sensor data representing the movement of the user's arm and the sensor data representing the occurrence of the sound to identify that the sound occurred shortly before the movement of the user's arm. The proximity of the occurrence of the sound to the movement of the user's arm can indicate that the sound likely caused the user to move his arm. Sensor data processor 161 can then store a correlation between the sound and the movement to indicate that the user likely moved in response to the sound.

In this way, a better indication of the user's sleep patterns can be provided. For example, the mobile device can track such correlations that may occur during the user's sleep and generate an analysis that indicates how much of the user's movement during sleep was likely caused by external or environmental factors such as sound or light. By having such an analysis, the user can know that any issues with his sleep patterns are not likely due to any internal problems the user may have, but are more likely a result of the external occurrences of sound, light, or other environmental occurrence detectable by a sensor on a mobile device.

In some cases, a correlation can be given a strength. For example, if the movement occurs immediately after or during a dog bark, a strong correlation can be indicated whereas a weaker correlation can be indicated as the duration between the dog bark and the movement increases. Similarly, the strength of the correlation can be based on how loud the dog bark was. For example, if the dog bark is loud, the strength of the correlation can be higher than when the dog bark is soft.

Similar strengths of the correlation can be created when the sensor data obtained from a sensor of the mobile device is from a light or other sensor. For example, the occurrence of a brighter light can result in a higher correlation strength than the occurrence of a dimmer light.

In addition to creating correlations between a user's movements and environmental occurrences, some embodiments of the present invention can also create correlations between the user's physiological parameters and an environmental occurrence. For example, a heart rate sensor within the wearable sensor device (or another wearable sensor device the user is wearing) can transmit the user's heart rate to the mobile device. When there is an environmental occurrence such as a sound or a light, the heart rate of the user at the time of the environment occurrence can be correlated with the environmental occurrence.

For example, if the mobile device identifies that the user's heart rate spikes at time t2 and a loud sound was audible at time t1, the mobile device can determine whether the duration between t1 and t2 indicates that the spike in the heart rate was likely due to the loud sound and create a correlation accordingly.

Because the platform of the present invention can allow the tracking and correlation of sensor data from both wearable sensor devices and mobile devices, the information that can be generated to represent the user's sleep patterns and activities can provide a more accurate indication of how the user is sleeping and why the user is performing certain actions during sleep. Without such correlations, the user is only informed of when the user moved but is not provided with any indication of why the user moved. This can cause the user to assume there is something wrong with his sleep patterns when in fact the problem is due only to external factors.

The techniques described above for correlating a user's actions during sleep with environmental occurrences can also be used to correlate a user's actions during an activity with sensor data generated by sensors of a mobile device. For example, one or more sensors of the mobile device can be used to generate sensor data during a user's activity which is correlated with sensor data generated by one or more wearable sensor devices worn by the user during the activity.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed:

1. One or more computer storage media storing a platform for facilitating the use of a mobile device for receiving raw sensor data from a wearable sensor device, the platform comprising:
a sensor data processor that executes on a mobile device which is configured to process raw sensor data into a usable format for consumption by other applications; and
a sensor data interface which executes on a wearable sensor device for receiving raw sensor data and transmitting the raw sensor data to a mobile device for processing by the sensor data processor.

2. The computer storage media of claim 1, wherein the platform further comprises:
a firmware generator the executes on the mobile device which can wirelessly update the firmware on the wearable sensor device to customize the functionality of the wearable sensor device for a particular use or user; and
a firmware updater that executes on the wearable sensor device to receive firmware updates and apply the updates to customize the functionality of one or more sensors or other components on the wearable sensor device.

3. The computer storage media of claim 1, wherein the platform further comprises:
a Bluetooth low energy (BLE) module for implementing the BLE protocol for communicating sensor data from the wearable sensor device to the mobile device.

4. The computer storage media of claim 1, wherein the platform further comprises:
a charging module on the wearable sensor device that enables inductive charging of a battery on the wearable sensor device without interfering with the functionality of any sensors on the wearable sensor device.

5. The computer storage media of claim 1, wherein the wearable sensor device includes one or more accelerometers, and the sensor data processor is configured to perform a method for identifying a particular movement from accelerometer data by comparing an identified sequence in the accelerometer data to known sequences, the method comprising:
storing a plurality of entries in a database, each entry representing one or more known sequences of accelerometer data that are generated when a particular movement is performed;
receiving accelerometer data from the sensor data interface of the wearable sensor device worn by a user while performing a first movement;
accessing the database to determine that the accelerometer data received from the sensor data interface includes the one or more known sequences of a first entry; and
determining that the first entry is associated with a first particular movement.

6. The computer storage media of claim 5, wherein the sensor data processor is further configured to output an indication that the first particular movement has been performed by the user.

7. The computer storage media of claim 6, wherein outputting the indication comprises incrementing a count of the number of times the user has performed the first particular movement.

8. The computer storage media of claim 1, wherein the sensor data processor is configured to create entries in a database that link raw sensor data received from the sensor data interface with one or more known movements.

9. The computer storage media of claim 8, wherein at least one entry comprises a sequence of accelerometer data.

10. The computer storage media of claim 2, wherein the firmware generator is configured to:
receive information about a particular user that uses the wearable sensor device;
determine, from the information about the particular user, that a first version of firmware installed on the wearable sensor device for controlling at least one sensor is not optimal;
generate a second version of the firmware, the second version of the firmware being customized, based on the information about the particular user, to control the at least one sensor in a more optimal manner; and
transmit the second version of the firmware to the firmware updater of the wearable sensor device.

11. The computer storage media of claim 10, wherein the second version of the firmware causes the at least one sensor to perform one or more of the following:
employ a different sampling rate;
employ a different power level; or
employ a different rate for transmitting raw sensor data to the sensor data processor.

12. The computer storage media of claim 2, wherein a firmware update comprises an updated value for one or more customizable parameters of a sensor.

13. The computer storage media of claim 1, wherein the sensor data processor comprises computer executable instructions that are downloadable to a mobile device to enable the mobile device to communicate with the sensor data interface.

14. The computer storage media of claim 1, wherein the sensor data processor is also configured to process raw sensor data received from one or more sensors located on the mobile device.

15. One or more computer storage media storing a platform for facilitating the use of a mobile device for receiving raw sensor data from a wearable sensor device, the platform comprising:
a sensor data processor that executes on a mobile device which is configured to process raw sensor data received from a wearable sensor device into a usable format for consumption by other applications, the raw sensor data comprising raw sensor data received from a plurality of different types of sensors.

16. The one or more computer storage media of claim 15, wherein the platform further comprises a firmware generator the executes on the mobile device which can wirelessly update firmware on the wearable sensor device to customize the functionality of one or more sensors of the wearable sensor device for a particular use or user.

17. The one or more computer storage media of claim 16, wherein the one or more sensors are different types of sensors such that the firmware generator can wirelessly update the firmware of multiple types of sensors.

18. One or more computer storage media storing a platform for facilitating the use of a mobile device for receiving raw sensor data from a wearable sensor device, the platform comprising:
a sensor data interface which executes on a wearable sensor device for receiving raw sensor data from a plurality of different types of sensors on the wearable sensor device and transmitting the raw sensor data to a sensor data processor executing on a mobile device.

19. The one or more computer storage media of claim 18, wherein the platform further comprises a firmware updater that executes on the wearable sensor device to receive firmware updates from a firmware generator executing on the mobile device and to apply the updates to customize the functionality of one or more sensors or other components on the wearable sensor device.

20. The one or more computer storage media of claim 18, wherein the platform further comprises one or more of:
a Bluetooth low energy (BLE) module for implementing the BLE protocol for communicating sensor data from the wearable sensor device to the mobile device; or
a charging module on the wearable sensor device that enables inductive charging of a battery on the wearable sensor device without interfering with the functionality of any sensors on the wearable sensor device.
